(19) United States
(12) Patent Application Publication
(10) Pub. No.: US 2024/0390669 A1
(43) Pub. Date: Nov. 28, 2024

(54) DEVICE FOR CURING RHINITIS

(71) Applicant: PROXI HEALTHCARE INC., Ulsan (KR)

(72) Inventor: Young Wook KIM, Seoul (KR)

(73) Assignee: PROXI HEALTHCARE INC., Ulsan (KR)

(21) Appl. No.: 18/287,297

(22) PCT Filed: Oct. 7, 2021
(86) PCT No.: PCT/KR2021/013755

§ 371 Date: Aug. 5, 2024

(30) Foreign Application Priority Data
Apr. 20, 2021 (KR) 10-2021-0050816

(51) Int. Cl. A61N 1/05; A61N 1/36

(52) U.S. Cl. CPC A61N 1/0546; A61N 1/36034

(57) ABSTRACT

Provided is a device for curing rhinitis. The device for curing rhinitis may include: a curing unit including a first insertion unit and a second insertion unit each of which is inserted into different nostrils of a user; a power equipment including a signal applying unit which supplies a driving signal to the first insertion unit and the second insertion unit; and a cable for connecting the curing unit to the power equipment, wherein the first insertion unit and the second insertion unit each include a first electrode and a second electrode which are spaced apart from each other, receive the driving signal, and provide electromagnetic wave that corresponds to the driving signal.

### TECHNICAL FIELD

The present invention relates to a device for curing rhinitis which uses microcurrent special electromagnetic waves.

### BACKGROUND ART

Rhinitis is a disease which signifies an inflammation within a nose (in nasal cavity) and is mainly divided into allergic rhinitis and chronic rhinitis. A clear criteria to divide both rhinitis does not exist. It is hard to identify that the rhinitis itself is a fatal illness that threatens life but it is hard to be completely recovered. The rhinitis may be a disease that significantly lowers quality of life.

Actually, in case of allergic rhinitis, a prevalent rate of 10-20% is shown in the West and a prevalent rate of 15% is shown in Korea. A prevalent rate of the allergic rhinitis is gradually increasing due to environmental changes such as air pollution, increased use of food additives, and urbanization. In this regard, it is important to manage the rhinitis in everyday life, to solve the basic cause of the rhinitis by improving living habits, and to relieve symptoms appeared by the rhinitis.

In general, as a rhinitis curing device which is used for the purpose of relieving and curing symptoms of rhinitis, products manufactured by using an ultrasonic spraying method and a compressed air spraying method are widely used, wherein in the ultrasonic spraying method, therapeutic medicine, for example, saline solution, benzethonium chloride, or alcohol, is used to make steam having tiny particles by using ultrasonic waves and the steam is sprayed, and in the compressed air spraying method, steam generated by heating therapeutic medicine or saline solution at a uniform temperature is expanded to eject compressed air and the compressed air is sprayed.

Here, in general, the compressed air spraying method is widely used, since air pressure is used to minutely and strongly spray therapeutic medicine and thereby, treatment effect is excellent compared with the ultrasonic spraying method.

However, in the rhinitis curing devices manufactured by using the ultrasonic spraying method and the compressed air spraying method, an excessive amount of medicine may be consumed at every time of use. Also, the rhinitis curing devices directly spray liquid drug, which is foreign substance, to the affected area within a nose. Accordingly, a stimulus is given to the inside of the nose due to a shock generated during spraying and thereby, other rhinitis diseases may be caused. In this regard, usability is lowered and thus, kids or aged people who relatively have many rhinitis diseases avoid the use of the rhinitis curing devices. In addition, as the rhinitis curing devices need to be placed at a certain spot, there are limitations in their use.

Recently, laser rhinitis curing devices have been developed by low level laser therapy (LLLT) in order to irradiate semiconductor laser to the nasal cavity and to improve allergic rhinitis symptoms by bio-stimulation effect of laser. However, a therapeutic range to which laser is irradiated is narrow due to straightness of laser and thereby, treatment effect is low.

### DETAILED DESCRIPTION OF THE INVENTION

### Technical Problem

The present invention provides a device for curing rhinitis which provides electromagnetic waves so as to expect rhinitis treatment effect.

The present invention also provides a device for curing rhinitis which uses a driving signal generated by mixing an AC signal with a DC signal so as to increase inflammation management effect.

### Technical Solution

According to an aspect of the present invention, there is provided a device for curing rhinitis including: a curing unit including a first insertion unit and a second insertion unit each of which is inserted into different nostrils of a user; a power equipment including a signal applying unit which supplies a driving signal to the first insertion unit and the second insertion unit; and a cable for connecting the curing unit to the power equipment, wherein the first insertion unit and the second insertion unit each comprise a first electrode and a second electrode which are spaced apart from each other, receive the driving signal, and provide electromagnetic wave that corresponds to the driving signal.

Also, the curing unit may further include a fixing body on which a pair of holes are formed so that the first insertion unit and the second insertion unit may penetrate and may be inserted thereinto.

Also, each of the first insertion unit and the second insertion unit may further include: a head on which the first electrode and the second electrode are each disposed; a head cover which is combined to the head and includes at least one opening to partially expose the electrodes; a body extended from the head; a first connection electrode and a second connection electrode which are disposed within the body and respectively connected to the first electrode and the second electrode; and a body cover which closes the end part of the body.

Also, the first electrode and the second electrode may each include a first end part and a second end part which are located in the opposite direction and the head may include a pair of first openings to expose the first end part and the second end part of the first electrode; and a pair of second openings to expose the first end part and the second end part of the second electrode.

The body cover may include a pillar unit disposed between the end part of the first connection electrode and the end part of the second connection electrode; and a pair of accommodating grooves which are disposed at both sides of the pillar unit and accommodate each end part of the first connection electrode and the second connection electrode.

The first insertion unit and the second insertion unit may respectively and further include: a first connection terminal and a second connection terminal which are disposed in the pair of accommodating grooves and respectively connected to the first connection electrode and the second connection electrode; and a first connection line and a second connection line which are respectively connected to the first connection terminal and the second connection terminal within the body cover and are partially exposed through the body cover.

The first electrode and the second electrode may respectively and further include a third end part which is one end of an electrode area protruded and extended upward from a center area and the head may include a pair of openings which are disposed on the upper surface thereof and expose the third end part of the first electrode and the third end part of the second electrode.

The driving signal may be generated by mixing an AC signal and a DC signal.

The device for curing rhinitis may further include a control unit for controlling the signal applying unit so that at least one of characteristics of the driving signal may change in response to users' control.

The characteristics of the driving signal may include an amplitude and a DC offset.

The characteristics of the driving signal supplied to the first insertion unit and the characteristics of the driving signal supplied to the second insertion unit may be set to be different from each other.

According to another aspect of the present invention, there is provided a device for curing rhinitis including: insertion units in which at least a part thereof may be inserted into nostrils of a user and which include at least one electrode for receiving a driving signal and providing electromagnetic wave that corresponds to the driving signal; and a signal applying unit which mixes an AC signal and a DC signal, generates the driving signal, and supplies the driving signal to the electrodes.

### Effects of the Invention

According to the present invention, a device for curing rhinitis may be provided, wherein the device provides electromagnetic waves and thereby, rhinitis treatment effect may be expected.

Also, a device for curing rhinitis may be provided, wherein the device uses a driving signal generated by mixing an AC signal and a DC signal and thereby, inflammation management effect increases.

### DESCRIPTION OF THE DRAWINGS

FIG. 1 illustrates a device for curing rhinitis according to an embodiment of the present invention;

FIGS. 2A and 2B are diagrams for describing biofilm removal effect of a driving signal generated by mixing an Alternating Current (AC) signal with a Direct Current (DC) signal;

FIGS. 3 and 4 illustrate a curing unit according to an embodiment of the present invention;

FIG. 5 illustrates a part of a first insertion unit from which a head cover is separated;

FIG. 6 illustrates a first insertion unit viewing from the top;

FIG. 7A illustrates a combination relation between electrodes, connection electrodes, connection terminals, and a body cover included in a first insertion unit according to an embodiment of the present invention;

FIG. 7B illustrates the body cover of FIG. 7A;

FIG. 8 illustrates a power equipment according to an embodiment of the present invention;

FIG. 9 illustrates an internal structure of a power equipment according to an embodiment of the present invention;

FIGS. 10A through 10C are graphs showing waveforms of signals according to an embodiment of the present invention; and

FIG. 11 illustrates an internal structure of a power equipment according to another embodiment of the present invention.

[DESCRIPTION OF THE REFERENCE NUMERALS]

| | |
|---|---|
| 10: curing unit | 11: electrode |
| 11a: first electrode | 11b: second electrode |
| 20: power equipment | 30: battery |
| 40: signal applying unit | 50: control unit |
| 60: cable | |

### BEST MODE OF THE INVENTION

Hereinafter, embodiments of the present invention will be described in more detail with reference to the accompanying drawings. It should be understood that the present invention may, however, be embodied in many different forms and should not be construed as being limited to the embodiments set forth herein; rather exemplary embodiments are to cover all modifications, equivalents, and alternatives falling within the scope of the invention.

It will be understood that, although the terms first, second, A, B, (a), (b), etc. may be used herein to describe various elements, these terms are only used to distinguish one element from another. Thus, properties, orders, or procedures of the corresponding elements are not limited by these terms. Also, it will be understood that when an element is referred to as being “connected to,” “combined to”, or “contact with” another element, it may be directly connected to or contact with the other element. However, intervening elements may exist between each element to connected to, combined to, or contact with the elements. It should be understood that “connection”, “combination” or “contact” refer to not only physical connection”, “combination” or “contact” but also electrical connection”, “combination” or “contact”.

The terms “unit”, “equipment”, “device”, “module”, and etc. used in this specification denote a unit for processing at least one function or operation and may be embodied in hardware, software, or a combination of hardware and software. It will be further understood that the terms “include” “form”, “have”, and etc. when used herein, specify the presence of stated elements, unless there is no opposition. Accordingly, the terms are not intended to exclude other elements and instead, are intended to further add the elements.

In addition, classification of elements in this specification is only to clearly present main functions of each element. That is, in the description below, at least two elements may be combined as one element or one element may be divided into at least two elements by each subdivided function. In addition, each of the elements hereinafter may further execute a part of or all functions from among functions of other elements, in addition to their own main functions. It may also available that a part of main functions executed by each element may be executed by other elements.

Hereinafter, a device for curing rhinitis according to an embodiment of the present invention will be described in more detail with reference to the accompanying drawings.

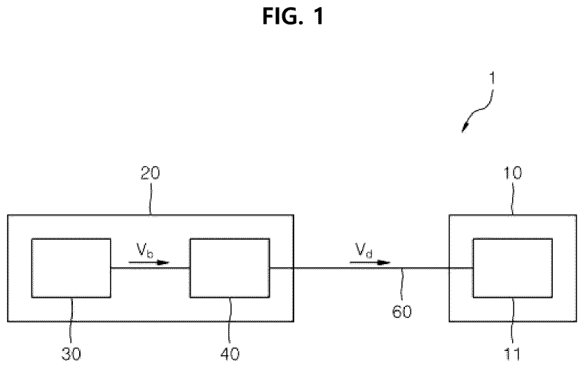

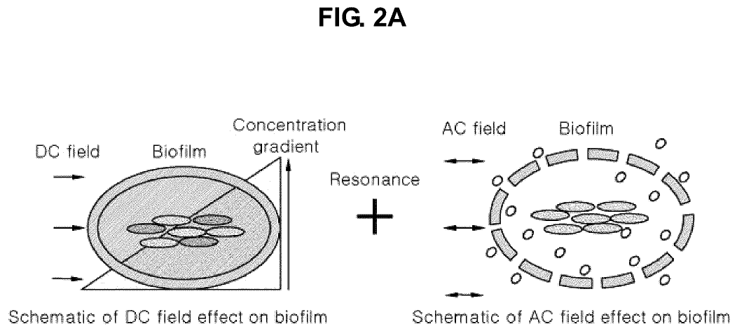

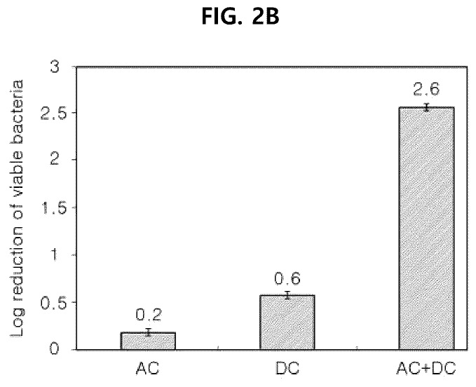

FIG. 1 schematically illustrates a device for curing rhinitis according to an embodiment of the present invention and FIGS. 2A and 2B are diagrams for describing biofilm removal effect of a driving signal generated by mixing an Alternating Current (AC) signal and a Direct Current (DC) signal.

Referring to FIG. 1, a device 1 for curing rhinitis according to an embodiment of the present invention may include a curing unit 10, a power equipment 20, and a cable 60 connecting the curing unit 10 with the power equipment 20.

The curing unit 10 may include an electrode 11 which receives a driving signal Vd from the power equipment 20 and generates an electric field that corresponds to the driving signal Vd.

The electrode 11 may provide an electromagnetic wave based on electric energy of the driving signal Vd, wherein the electromagnetic wave has an electrical influence on a user and thereby, may provide the effects such as pain relief, a decrease in inflammation, and relief from sinus infection through biofilm removal.

When the electrode 11 is disposed in the curing unit 10 in a plural number, at least one electrode 11 from among the plurality of the electrodes 11 may receive the driving signal Vd. Here, an electrode functioning as a ground electrode may be further included in the curing unit 10.

The electrode 11 may be formed of a material including brass, aluminum, conducting polymer, conducting silicon, and stainless steel. However, the present invention is not limited thereto and any material having conductivity may be used as a material for forming an electrode.

The curing unit 10 may include a separate groove or hole formed therein and the electrode 11 may be inserted into the groove or hole. Also, a part of the electrode 11 may be exposed from the curing unit 10. Here, the electrode 11 may not be protruded to the outside of the curing unit 10 or may be protruded to the outside of the curing unit 10.

The power equipment 20 may include a battery 30 and a signal applying unit 40, wherein the signal applying unit 40 may generate the driving signal Vd by using a battery voltage Vb supplied from the battery 30.

In particular, the signal applying unit 40 generates the driving signal Vd by mixing an alternating current (AC) signal with a direct current (DC) signal.

In this regard, the driving signal Vd includes both AC ingredient and DC ingredient and thereby, a synergy effect and resonance created by simultaneously applying the AC ingredient and the DC ingredient are generated. Thus, the removal effect of a biofilm which is the cause of inflammation may be increased.

Referring to FIG. 2A, an electric field by the DC ingredient induces a local imbalance in distribution of electric charges and thereby, a structural stress of a biofilm increases. Also, an electric field by the AC ingredient may increase permeability of an external protector by generating specific vibration.

The synergy effect from such AC ingredient and DC ingredient may be identified in FIG. 2B. That is, compared with the biofilm removal effect when the electric field by the AC ingredient and the electric field by the DC voltage are each independently provided, the biofilm removal effect is significantly excellent when the electric fields by the AC ingredient and the DC voltage are overlapped and simultaneously provided.

The electric fields by the AC ingredient and the DC ingredient may be simultaneously provided from the electrode 11 according to the driving signal Vd supplied by the signal applying unit 40 according to an embodiment of the present invention and thereby, amplified removal effect for the biofilm may be accomplished.

Also, as described above, the driving signal Vd is set as a form in which an alternating current voltage and a direct current voltage are overlapped. Accordingly, compared with a case where a direct current voltage is only applied, an electric shock hazard to the body and pain caused to the body may be reduced.

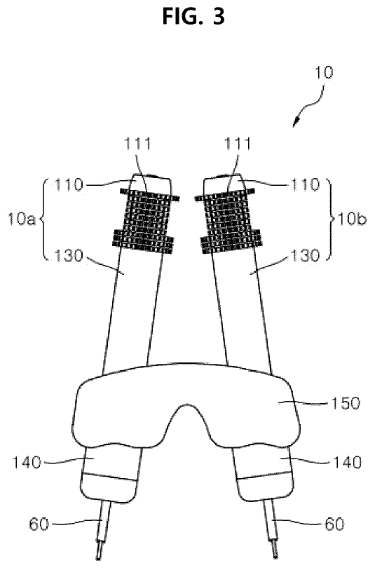

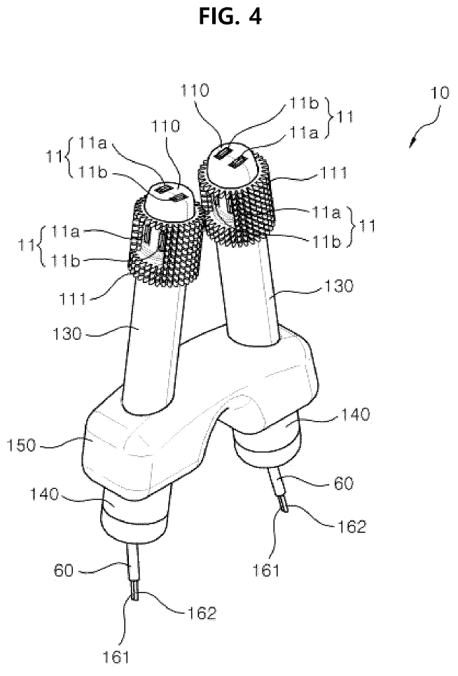

FIGS. 3 and 4 illustrate the curing unit 10 according to an embodiment of the present invention.

Referring to FIGS. 3 and 4, the curing unit 10 according to an embodiment of the present invention may include a pair of insertion units including a first insertion unit 10A and a second insertion unit 10B, and a fixing body 150 for fixing the pair of insertion units 10A and 10B.

The pair of insertion units 10A and 10B may be installed to be combined to and removed from the fixing body 150. In this regard, when the insertion units 10A and 10B and/or the fixing body 150 need to be replaced due to deterioration, a user may easily replace the existing insertion units 10A and 10B and/or fixing body 150 with new insertion units 10A and 10B and/or fixing body 150.

The first insertion unit 10A and the second insertion unit 10B may each be inserted into different nostrils. For example, the first insertion unit 10A may be partially inserted into a right nostril and the second insertion unit 10B may be partially inserted into a left nostril.

The first insertion unit 10A and the second insertion unit 10B may be formed of synthetic resins, wherein the synthetic resins partially contains graphene so as to implement antibiosis, far-infrared radiation release, and water repellency.

The fixing body 150 may include a pair of holes through which the first insertion unit 10A and the second insertion unit 10B penetrate and are inserted.

Also, the fixing body 150 may have an elastic force and thereby, may be formed of an elastic material. For example, when the curing unit 10 is inserted into a user's nose, the fixing body 150 is modified in response to a form of a user's nose and thereby, a gap between the first insertion unit 10A and the second insertion unit 10B may be changed. When the curing unit 10 is removed from a user's nose, the modified fixing body 150 may be restored to an original state.

As illustrated in the drawings, when the curing unit 10 is inserted into a user's nose, in order to facilitate movement of the first insertion unit 10A and the second insertion unit 10B that correspond to a form of a user's nose, a thickness of an area between the first insertion unit 10A and the second insertion unit 10B may be smaller than a thickness of an area where the first insertion unit 10A and the second insertion unit 10B are combined within the fixing body 150.

The first insertion unit 10A and the second insertion unit 10B may each include a head 110 and a body 130.

The head 110 may include a head cover 111 and the electrodes 11, wherein the electrodes 11 may be each disposed in one side surface or both side surfaces of the upper part of the head 110. Also, in order to smoothly provide electromagnetic wave, the electrodes 11 may be further disposed in the upper side surface of the head 110. The electrode 11 may include a first electrode 11A and a second electrode 11B. The first electrode 11A and the second electrode 11B are spaced apart from each other and are disposed in the inside of the head 110. A part of the first electrode 11A and the second electrode 11B may be exposed from the head 110. In this regard, the head 110 may have openings for the first electrode 11A and the second electrode 11B to be partially exposed.

The first electrode 11A and the second electrode 11B may provide electromagnetic wave by the driving signal Vd supplied from the signal applying unit 40.

Here, a gap between the first electrode 11A and the second electrode 11B included in any one insertion unit (for example, the first insertion unit 10A) may be 0.1 mm through 20 mm. When the gap between the first electrode 11A and the second electrode 11B is set to be below 0.1 mm or above 20 mm, the biofilm removal effect by the electromagnetic wave generated from the first electrode 11A and the second electrode 11B may not be expected.

The first electrode 11A and the second electrode 11B may have a size appropriate to be disposed within the head 110 having a size appropriate for users to insert their nostrils. For example, the width of the first electrode 11A recognized by viewing the device 1 for curing rhinitis at the front may be 0.1 mm through 10 mm and the length of the first electrode 11A may be 0.1 mm through 50 mm. Also, the second electrode 11B may be formed to have the same size with that of the first electrode 11A.

Meanwhile, when the width and the length of the first electrode 11A and the second electrode 11B are set to be below 0.1 mm, the management effect on the nasal cavity by an electric field may be lowered.

According to an embodiment of the present invention, the first electrode 11A and the second electrode 11B may be partially protruded from the head 110. In this case, the first electrode 11A may be protruded by a height of 0.1 mm through 15 mm from the surface of the head 110. Here, the second electrode 11B may be set to have the same height with that of the first electrode 11A.

When the height of the first electrode 11A and the second electrode 11B are set be below 0.1 mm, the first electrode 11A and the second electrode 11B may be buried in the inside of the head 110 while an insert injection molding process.

The body 130 may be extended from the head 110 to form a body of the curing unit 10. The body 130 may be designed to have an appropriate length for its use and the end part of the body 130 may be combined to the fixing body 150. The body 130 may be separately formed and combined to the head 110, or the body 130 and the head 110 may be formed as one body.

The body 130 may include a body cover 140 which is combined to the end part of the body 130 and closes the end part of the body 130. At least a part of the body cover 140 may be combined to the fixing body 150. That is, the end part of the body 130 and a part of the body cover 140 are inserted into the hole formed on the fixing body 150 and thereby, may be combined to the fixing body 150.

A first connection line 161 and a second connection line 162 may be partially exposed to the outside through the body cover 140.

As illustrated in FIG. 4, the first connection line 161 and the second connection line exposed to the outside of the body cover 140 are included in the cable 60 and are electrically connected to the power equipment 20 so that the driving signal Vd supplied from the power equipment 20 may be provided to the first electrode 11A and the second electrode 11B.

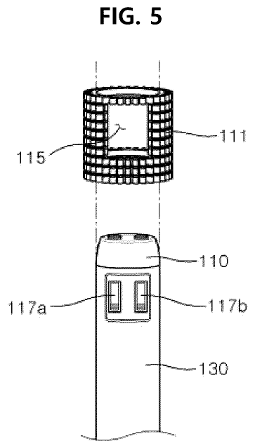

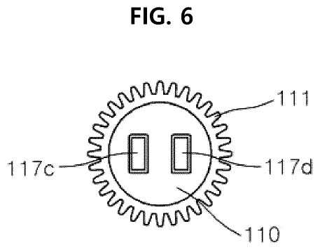

FIG. 5 illustrates the head cover 111 and a part of the first insertion unit 10A from which the head cover 111 is separated and FIG. 6 illustrates the first insertion unit 10A viewing from the top. Meanwhile, for convenience of description, the first electrode 11A and the second electrode 11B are not illustrated in FIGS. 5 and 6. Also, for convenience of description, the first insertion unit 10A is only illustrated in FIGS. 5 and 6 and the second insertion unit 10B may have the same form with that of the first insertion unit 10A.

Referring to FIGS. 5 and 6, the head cover 111 may be designed to be combined to and removed from the head 110. In this regard, when the head cover 111 needs to be replaced due to deterioration, a user may easily replace the existing head cover 111 with a new head cover 111.

Also, the head cover 111 may have an opening 115 for the first electrode 11A and a part of the second electrode 11B to be exposed. One end of the first electrode 11A and one end of the second electrode 11B may all be exposed through one opening 115. Here, the head cover 111 may include a pair of openings 115 which face each other so that the first electrode 11A and the second electrode 11B may be exposed to the both sides of the head 110.

In addition, the head cover 111 may include an opening on the upper surface thereof so that the first electrode 11A and the second electrode 11B may be exposed to the upper surface of the head 110.

The head cover 111 may be formed of silicon. However, the present invention is not limited thereto and the head cover 111 may be formed of various soft materials.

Next, the head 110 may include a pair of first openings 117A and a pair of second openings 117B, wherein the first electrode 11A is partially exposed to the pair of first openings 117A and the second electrode 11B is partially exposed to the pair of second openings 117B. In this case, the pair of first openings 117A may be formed to face each other and the pair of second openings 117B may be also formed to be face each other.

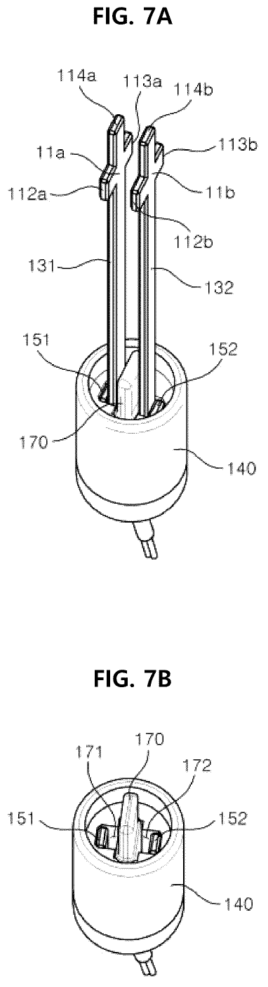

A first end part 112A (refer to FIG. 7A) of the first electrode 11A may be exposed to any one of the pair of first openings 117A and a second end part 113A (refer to FIG. 7A) of the first electrode 11A may be exposed to one remaining first opening 117A.

Furthermore, a first end part 112B (refer to FIG. 7A) of the second electrode 11B may be exposed to any one of the pair of second openings 117B and a second end part 113B (refer to FIG. 7A) of the second electrode 11B may be exposed to one remaining second opening 117B.

The head 110 may include a third opening 117C and a fourth opening 117D on the upper surface thereof. A third end part 114A (refer to FIG. 7A) of the first electrode 11A and a third end part 114B (refer to FIG. 7A) of the second electrode 11B may be respectively exposed to the third opening 117C and the fourth opening 117D.

More specifically, the pair of first openings 117A which face each other on the side surface of the head 110, the pair of second openings 117B which face each other on the side surface of the head 110 and are disposed in parallel with the first opening 117A, and third and fourth openings 117C and 117D which are adjacent to each other on the upper surface of the head 110 are included, the first electrode 11A and the second electrode 11B may be each exposed to the three openings.

FIG. 7A illustrates a combination relation between the electrodes 11A and 11B, connection electrodes 131 and 132, connection terminals 151 and 152, and the body cover 140 included in the first insertion unit 10A according to an embodiment of the present invention and FIG. 7B illustrates the body cover 140 of FIG. 7A. Meanwhile, for convenience of description, FIGS. 7A and 7B only illustrates the first insertion unit 10A including the electrodes 11A and 11B, the connection electrodes 131 and 132, the connection terminals 151 and 152, and the body cover 140. The electrodes, connection electrodes, connection terminals, and a body cover included in the second insertion unit 10B may be the same as those illustrated in FIG. 7A.

Referring to FIG. 7A, the first electrode 11A may include the first end part 112A, the second end part 113A, and the third end part 114A. The first end part 112A and the second end part 113A may be located in the opposite direction and the third end part 114A may be interposed between the first end part 112A and the second end part 113A.

More specifically, the first end part 112A may be one end of an electrode area which is protruded and extended in one direction from a center area of the first electrode 11A, the second end part 113A may be one end of an electrode area which is protruded and extended in other direction opposite to the one direction from a center area of the first electrode 11A, and the third end part 114A may be one end of an electrode area which is protruded and extended upward (a direction vertically to the one direction and other direction opposite to the one direction) from a center area of the first electrode 11A. Here, the center area of the first electrode 11A may be connected to one end of a first connection electrode 131 which will be described below.

The first end part 112A and the second end part 113A may be exposed to the pair of first openings 117A included in the head 110 and the third end part 114A may be exposed to the third opening 117C included in the head 110.

The second electrode 11B is formed as in the same form as the first electrode 11A and includes the first end part 112B and the second end part 113B located in the opposite direction, wherein the first end part 112B and the second end part 113B may be exposed to the pair of second openings 117B included in the head 110. Also, the second electrode 11B may be interposed between the first end part 112B and the second end part 113B and may include the third end part 114B exposed to the fourth opening 117D included in the head 110.

That is, at least a part of the first electrode 11A and the second electrode 11B may have a ‘T’ shape.

Next, the body 130 may include the first connection electrode 131 and a second connection electrode 132 arranged therein to transmit the driving signal Vd.

The first connection electrode 131 and the second connection electrode 132 may be respectively connected to the first electrode 11A and the second electrode 11B. More specifically, one end of the first connection electrode 131 may be connected to the center area of the first electrode 11A and one end of the second connection electrode 132 may be connected to the center area of the second electrode 11B.

Also, in order to receive power from the power equipment 20, the first connection electrode 131 and the second connection electrode 132 are extended long toward the end part of the body 130 and thereby, may be electrically connected to a first connection terminal 151 and a second connection terminal 152 disposed in the body cover 140. Specifically, the end part of the first connection electrode 131 may be connected to the first connection terminal 151 and the end part of the second connection electrode 132 may be connected to the second connection terminal 152.

For convenience of the manufacture, the first connection electrode 131 and the first electrode 11A may be formed as one body and the second connection electrode 132 and the second electrode 11B may be formed as one body.

Also, the first connection electrode 131 and the second connection electrode 132 may be formed of a material that is same as that of the electrodes described above.

In the body cover 140, the first connection terminal 151 and the second connection terminal 152 may be respectively connected to the first connection line 161 and the second connection line 162, wherein the first connection line 161 and the second connection line 162 may be partially exposed to the outside of the body cover 140.

The body cover 140 may include a pillar unit 170, wherein the pillar unit 170 may be disposed at the center of the body cover 140 and disposed between the end part of the first connection electrode 131 and the end part of the second connection electrode 132.

The pillar unit 170 may function as a spacer in such a way that the first connection electrode 131 and the second connection electrode 132 are spaced apart from each other by the pillar unit 170 and are prevented from contacting and being electrically connected to each other.

Referring to FIG. 7B, the pillar unit 170 may have a pair of accommodating grooves including a first accommodating groove 171 and a second accommodating groove 172 formed at both sides thereof, wherein the first accommodating groove 171 may accommodate at least a part of the first connection electrode 131 and the first connection terminal 151, and the second accommodating groove 172 may accommodate at least a part of the second connection electrode 132 and the second connection terminal 152.

That is, the end part of the first connection electrode 131 may contact the first connection terminal 151 within the first accommodating groove 171 and the end part of the second connection electrode 132 may contact the second connection terminal 152 within the second accommodating groove 172.

In the first insertion unit 10A, other elements except the body cover 140 and the connection terminals 151 and 152 and the connection lines 161 and 162 included in the body cover 140, that is, the head 110, the body 130, the electrodes 11A and 11B, and the connection electrodes 131 and 132 may be designed to be replaceable.

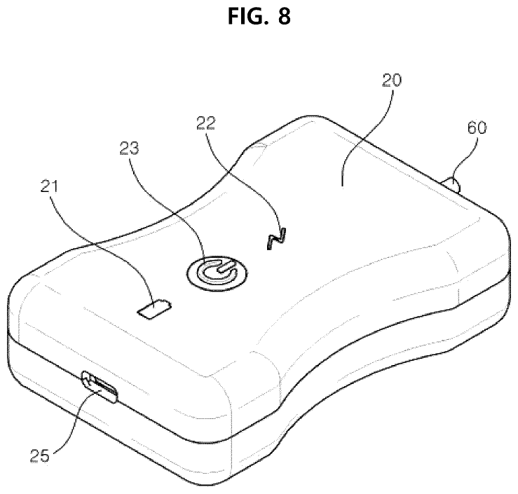

FIG. 8 illustrates the power equipment 20 according to an embodiment of the present invention.

Referring to FIG. 8, the power equipment 20 according to an embodiment of the present invention may include a first display unit 21, a second display unit 22, a switch 23, and a charging terminal 25.

The first display unit 21 may display the charge state of a battery disposed within the power equipment 20. For example, the first display unit 21 may include Light-Emitting Diode (LED) and may radiate a number of colors showing the charge state (for example, red) and the full-charge state (for example, blue).

The second display unit 22 may display the operating time of the device 1 for curing rhinitis. For example, the second display unit 22 may include LED and radiate a number of colors displaying 15 minute operation (for example, green) and 30 minute operation (for example, yellow).

The switch 23 may control power on/off of the device 1 for curing rhinitis and may be used to set the operating time.

The charging terminal 25 may be used to charge the battery disposed within the power equipment 20.

Meanwhile, in the power equipment 20, the cable 60 may be connected to an output terminal used to supply the driving signal and to combine with the curing unit 10. The output terminal may be electrically connected to the first connection line 161 and the second connection line 162.

The power equipment 20 may generate a driving signal by using a battery voltage supplied from the battery disposed therein and may supply the generated driving signal to the electrodes 11A and 11B through the first connection line 161 and the second connection line 162 connected to the output terminal.

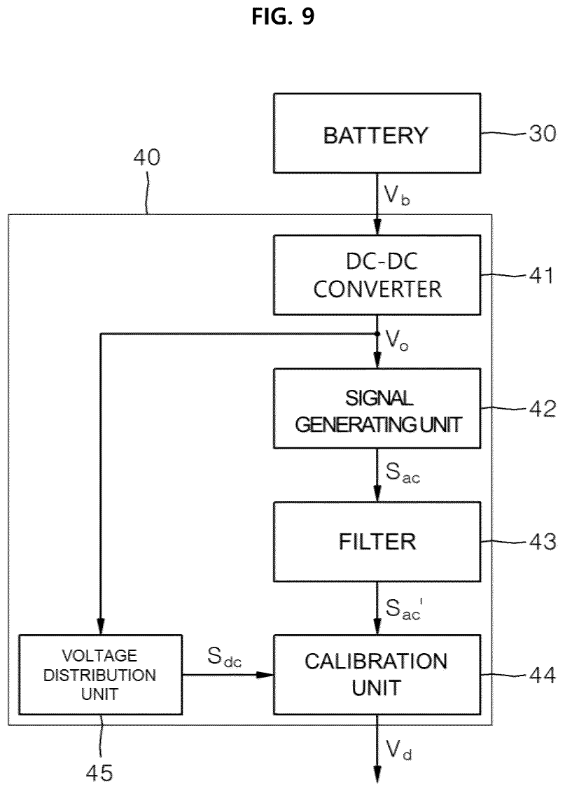

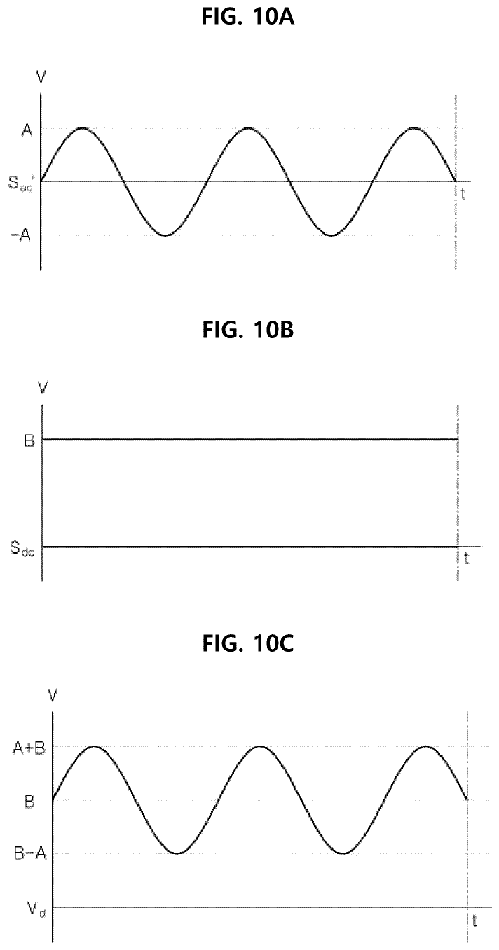

FIG. 9 illustrates an internal structure of the power equipment 20 according to an embodiment of the present invention and FIGS. 10A through 10C are graphs showing waveforms of signals according to an embodiment of the present invention. In particular, FIG. 10A illustrates a filtered AC signal Sac′, FIG. 10B illustrates a DC signal Sdc, and FIG. 10C illustrates the driving signal Vd generated by mixing the filtered AC signal Sac′ and the DC signal Sdc.

Referring to FIG. 9, the power equipment 20 may include the battery 30 and the signal applying unit 40.

The battery 30 included within the power equipment 20 may supply the battery voltage Vb to the signal applying unit 40.

For example, the battery 30 may be set as a primary battery or a secondary battery.

When the battery 30 is a primary battery, a user may periodically replace the battery 30. When the battery 30 is a secondary battery, charging may be accomplished through various charging methods. For example, wired charging may be accomplished through the charging terminal 25 described above or wireless charging may be also accomplished.

Next, the signal applying unit 40 according to an embodiment of the present invention may include a DC-DC converter 41, a signal generating unit 42, a filter 43, and a calibration unit 44. In addition, a voltage distribution unit 45 may be further included.

The DC-DC converter 41 may receive the battery voltage Vb from the battery 30, may convert the battery voltage Vb into an output voltage Vo in a predetermined level, and may output the output voltage Vo.

The signal generating unit 42 operates based on a voltage supplied from the DC-DC converter 41 and may generate an AC signal Sac having a predetermined frequency by using the output voltage Vo of the DC-DC converter 41.

The signal generating unit 42 may be embodied by using a well-known component such as an oscillator and a function generator which may generate the AC signal.

For example, the AC signal Sac may be set to have a frequency of 1 KHz through 1000 MHz. When the AC signal Sac is set to have a low frequency of below 1 KHz, the inflammation relief effect through biofilm removal is decreased. When the AC signal Sac is set to have a super-high frequency of above 1000 MHz, the biofilm removal effect is also decreased. Meanwhile, the frequency of the AC signal Sac may be set to 5 MHz through 15 MHz which is appropriate to remove a biofilm.

Also, an amplitude of the Ac signal Sac may be set to 0.1 mv through 3V. When the amplitude of the Ac signal Sac is below 0.1 mV, the biofilm removal effect may not be expected. When the amplitude of the Ac signal Sac is above 3V, toxic substances may be generated due to electrolysis of body fluids.

The filter 43 may perform a filtering process for the AC signal Sac generated by the signal generating unit 42. For example, the filter 43 may include a low pass filter and may convert the AC signal Sac in a sawtooth wave form into the AC signal Sac′ in a sine wave form. However, the type of the filter 43 is not particularly restricted thereto and various types of filter may be employed according to a designed structure.

The calibration unit 44 may mix the AC signal Sac′ supplied through the filter 43 with the DC signal Sdc to generate the driving signal Vd. For example, the calibration unit 44 may be embodied as an operating amplifier which may add (or overlap) the AC signal Sac′ and the DC signal Sdc, however, the present invention is not limited thereto.

In this regard, offset that corresponds to the DC signal Sdc is generated in the Ac signal Sac′ and thereby, the driving signal Vd having both AC component and DC component may be generated.

Since the driving signal Vd includes all characteristics of the AC signal Sac, the driving signal Vd may be set to have a frequency of 1 KHz through 1000 MHz and may be also set to have a frequency of 5 MHz through 15 MHz, which is more appropriate to remove the biofilm. In addition, an amplitude of the driving signal Vd may be set to 0.1 mv through 3V.

Referring to FIG. 10A, the calibration unit 44 may receive the AC signal Sac′ having the amplitude of A volt V from the filter 32. The DC signal Sdc having the amplitude of B volt V illustrated in FIG. 10B is overlapped with the AC signal Sac′ and thereby, the final driving signal Vd may be generated as illustrated in FIG. 10C.

Here, a voltage value of the DC signal Sdc may be set to above the amplitude of the AC signal Sac′. Accordingly, the voltage value of the driving signal Vd may be set to be above 0.

Finally, a DC offset value of the driving signal Vd may be the same as or greater than the amplitude of the driving signal Vd.

When the DC offset value of the driving signal Vd is below the amplitude of the driving signal Vd, a section where the driving signal Vd has a negative value is generated. Here, since the voltage has a negative value in the section, a loss of electric energy may occur.

However, as in an embodiment of the present invention, when the DC offset value of the driving signal Vd is set to be the same as or greater than the amplitude of the driving signal Vd, the voltage of the driving signal Vd is always above 0 and thereby, a loss of electric energy may be minimized.

Meanwhile, the DC signal Sdc may be generated by the voltage distribution unit 45. For example, the voltage distribution unit 45 may receive the output voltage Vo from the DC-DC converter 41 and may perform voltage distribution for the output voltage Vo so as to generate the DC signal Sdc.

The voltage distribution unit 45 may include a resistant string for distributing the output voltage Vo, however, is not restricted thereto.

When the output voltage Vo of the DC-DC converter 41 is appropriate to be immediately used to generate the driving signal Vd, the corresponding output voltage Vo may take a role of the DC signal Sdc. In this case, the voltage distribution unit 45 may be omitted and the output voltage Vo of the DC-DC converter 41 may be input to the calibration unit 44.

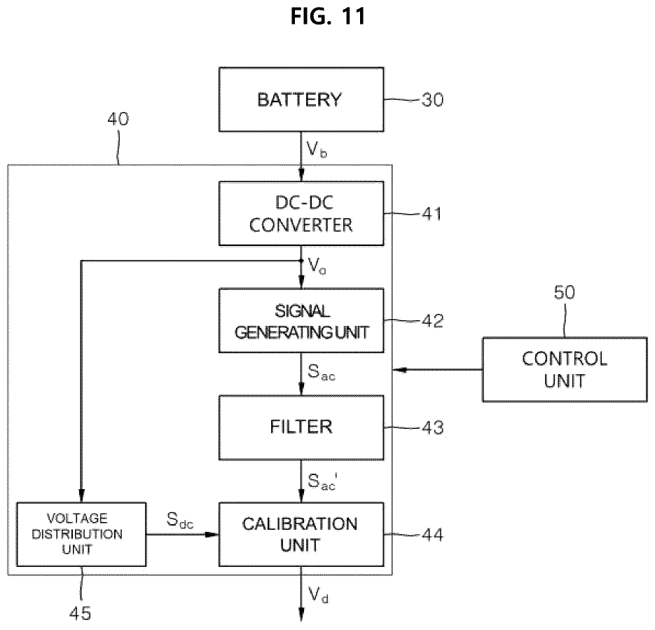

FIG. 11 illustrates an internal structure of the power equipment 20 according to another embodiment of the present invention.

Referring to FIG. 11, in the power equipment 20 according to another embodiment of the present invention, the signal applying unit 40 may change at least one of the characteristics of the driving signal Vd in response to a users' control. Also, the power equipment 20 may further include a control unit 50 for controlling the signal applying unit 40 in response to user's input.

For example, the characteristics of the driving signal Vd may include the amplitude of the driving signal Vd and the DC offset.

That is, users may control at least one of the amplitude of the driving signal Vd and the DC offset and thereby, may set optimal driving signal Vd that is appropriate to users. According to such function, customized rhinitis curing, in which users' characteristics are considered, may be available.

Here, users' input for controlling the characteristics of the driving signal Vd may be performed by the switch 23 installed in the power equipment 20 or a separate button (not illustrated) and users control the switch 23 installed in the power equipment 20 so as to control or set the characteristics of the driving signal Vd.

When setting information of the driving signal Vd is input by operating the switch from the user, the control unit 50 may control the signal applying unit 40 so that the driving signal Vd has the amplitude and the DC offset value that correspond to the input setting information.

The control unit 50 controls the signal generating unit 42 and thereby, may change the amplitude of the AC signal Sac. Also, the control unit 50 controls the DC-DC converter 41 and/or the voltage distribution unit 45 and thereby, may control the voltage value of the DC signal Sdc. Accordingly, characteristics of the driving signal Vd may be finally changed.

Here, the control unit 50 may control the voltage distribution unit 45 for the voltage value of the DC signal Sdc to be set above the amplitude of the AC signal Sac′ and thereby, the voltage value of the driving signal Vd may be set to be above 0.

Meanwhile, according to an embodiment of the present invention, characteristics of the driving signal Vd supplied to the first insertion unit 10A and characteristics of the driving signal Vd supplied to the second insertion unit 10B may be set to be same as each other. That is, one driving signal Vd output from the signal applying unit 40 may be input to the first insertion unit 10A and the second insertion unit 10B.

According to another embodiment of the present invention, characteristics of the driving signal Vd supplied to the first insertion unit 10A and characteristics of the driving signal Vd supplied to the second insertion unit 10B may be set to be different from each other.

In this case, although not illustrated in the drawing, two signal applying units 40 are included, wherein one signal applying unit 40 may generate and output the driving signal Vd supplied to the first insertion unit 10A and the other signal applying unit 40 may generate and output the driving signal Vd supplied to the second insertion unit 10B.

In this regard, users may input setting information of the driving signal that corresponds to the first insertion unit 10A and setting information of the driving signal that corresponds to the second insertion unit 10B each differently and the control unit 50 may control each of the signal applying units 40.

Those of ordinary skill in the art to which the present invention pertains will understand that other concrete forms are capable of being made to the above-described embodiments without changing the technical ideas or essential features of the present invention. Therefore, it should be understood that there is no intent to limit exemplary embodiments to the particular forms disclosed. The scope of the present invention is defined by the following claims rather than the detailed description and it should be construed that all changes or modifications drawn from the meaning of the scope, the scope, and their equivalents in the claims are included in the scope of the present invention.

1. A device for curing rhinitis comprising:
a curing unit comprising a first insertion unit and a second insertion unit each of which is inserted into different nostrils of a user;
a power equipment comprising a signal applying unit which supplies a driving signal to the first insertion unit and the second insertion unit; and
a cable for connecting the curing unit to the power equipment,
wherein the first insertion unit and the second insertion unit each comprise a first electrode and a second electrode which are spaced apart from each other, receive the driving signal, and provide electromagnetic wave that corresponds to the driving signal.

2. The device for curing rhinitis of claim 1, wherein the curing unit further comprises a fixing body on which a pair of holes are formed so that the first insertion unit and the second insertion unit penetrate and are inserted thereinto.

3. The device for curing rhinitis of claim 2, wherein each of the first insertion unit and the second insertion unit further comprises:
a head on which the first electrode and the second electrode are each disposed;
a head cover which is combined to the head and comprises at least one opening to partially expose the electrodes;
a body extended from the head;
a first connection electrode and a second connection electrode which are disposed within the body and respectively connected to the first electrode and the second electrode; and
a body cover which closes the end part of the body.

4. The device for curing rhinitis of claim 3, wherein the first electrode and the second electrode each comprise a first end part and a second end part which are located in the opposite direction and the head comprises a pair of first openings to expose the first end part and the second end part of the first electrode; and a pair of second openings to expose the first end part and the second end part of the second electrode.

5. The device for curing rhinitis of claim 3, wherein the body cover comprises:
a pillar unit disposed between the end part of the first connection electrode and the end part of the second connection electrode; and
a pair of accommodating grooves which are disposed at both sides of the pillar unit and accommodate each end part of the first connection electrode and the second connection electrode.

6. The device for curing rhinitis of claim 5, wherein the first insertion unit and the second insertion unit respectively and further comprise:
a first connection terminal and a second connection terminal which are disposed in the pair of accommodating grooves and respectively connected to the first connection electrode and the second connection electrode; and
a first connection line and a second connection line which are respectively connected to the first connection terminal and the second connection terminal within the body cover and are partially exposed through the body cover.

7. The device for curing rhinitis of claim 4, wherein the first electrode and the second electrode respectively and further comprise a third end part which is one end of an electrode area protruded and extended upward from a center area and the head comprises a pair of openings which are disposed on the upper surface thereof and expose the third end part of the first electrode and the third end part of the second electrode.

8. The device for curing rhinitis of claim 1, wherein the driving signal is generated by mixing an AC signal and a DC signal.

9. The device for curing rhinitis of claim 1, further comprising a control unit for controlling the signal applying unit so that at least one of characteristics of the driving signal may change in response to users' control.

10. The device for curing rhinitis of claim 9, wherein the characteristics of the driving signal comprise an amplitude and a DC offset.

11. The device for curing rhinitis of claim 10, wherein the characteristics of the driving signal supplied to the first insertion unit and the characteristics of the driving signal supplied to the second insertion unit are set to be different from each other.

12. A device for curing rhinitis comprising:
insertion units in which at least a part thereof may be inserted into nostrils of a user and which comprise at least one electrode for receiving a driving signal and providing electromagnetic wave that corresponds to the driving signal; and
a signal applying unit which mixes an AC signal and a DC signal, generates the driving signal, and supplies the driving signal to the electrodes.
